(19) United States
(12) Patent Application Publication
(10) Pub. No.: US 2015/0344520 A1
(43) Pub. Date: Dec. 3, 2015

(54) CHROMATOGRAPHY MEDIA AND PROTEIN PURIFICATION METHOD USING THE SAME

(71) Applicant: JNC CORPORATION, Tokyo (JP)

(72) Inventors: Yoshihiro MATSUMOTO, Kumamoto (JP); Yasuto UMEDA, Kumamoto (JP); Shigeyuki AOYAMA, Tokyo (JP)

(21) Appl. No.: 14/720,991

(22) Filed: May 26, 2015

(30) Foreign Application Priority Data
May 27, 2014 (JP) 2014-108831

(51) Int. Cl. C07K 1/22; B01J 20/286

(52) U.S. Cl. CPC C07K 1/22; B01J 20/286

(57) ABSTRACT

A chromatography media having excellent salt tolerance, adsorption characteristics and so forth is provided. The chromatography media contains a base media involving porous particles and polyamine bonded with the base media, in which 20 to 40% of amino groups in the polyamine is modified with a hydrophobic group.

### CROSS REFERENCE TO RELATED APPLICATIONS

This application claims the priority benefits of Japanese application serial no. 2014-108831, filed on May 27, 2014. The entirety of the above-mentioned patent application is hereby incorporated by reference herein and made a part of this specification.

### TECHNICAL FIELD

The present invention relates to a chromatography media, and a protein purification method using the same.

### BACKGROUND ART

Purifying a biomedicine applying chromatography is widely known, and separation of impurities from an object is performed utilizing various kinds of intermolecular interactions. Specific examples include a separation method utilizing an electrostatic interaction in ion exchange chromatography, a separation method utilizing a hydrophobic interaction in hydrophobic chromatography, and a separation method utilizing an affinity interaction to an antibody, such as protein A chromatography.

A technique most frequently applied in purification of the biomedicine is the ion exchange chromatography. In the ion exchange chromatography, adsorption performance is well known to decrease by electrical conductivity of a treatment liquid increasing. Accordingly, in a sample having high electrical conductivity, the electrical conductivity needs to be decreased by dilution or salt removal before applying adsorption treatment.

In order to compensate such a disadvantage of the ion exchange chromatography, a chromatography media concurrently having the hydrophobic interaction, a hydrophilic interaction, a chelate interaction and so forth, in addition to the electrostatic interaction, has been actively developed in recent years.

Thus, as the chromatography media concurrently having a plurality of actions, such a media is commercially available as Capto (registered trademark) adhere, Capto (registered trademark) MMC (both, made by GE Healthcare Ltd.), MEP HyperCel, HyperCel (trademark) AX STAR (all, made by Pall Corporation), Eshumuno (registered trademark) HCX (made by EMD Millipore Corporation), CHT (registered trademark) Ceramic Hydroxyapatite (made by Bio-Rad Laboratories, Inc.) and Toyopearl (registered trademark) MX Trp-650 (made by TOSOH Corporation).

The above chromatography media concurrently having the plurality of actions is obtained by introducing into an identical base media a ligand having an interaction according to a different principle, in addition to a ligand having the electrostatic interaction. The thus obtained media has selectivity different from the selectivity of the ion exchange chromatography media prepared by mediating only the ligand having the electrostatic interaction. Moreover, a media exhibiting good adsorption characteristics under conditions close to the conditions of a cell culture supernatant (for example, under conditions of the electrical conductivity comparable to the electrical conductivity of the culture supernatant) is also known.

For example, Non-patent literature No. 1 indicates that, in purification of a monoclonal antibody, a chromatography media concurrently having a plurality of actions is superior to an ion exchange chromatography media in removal of impurities, particularly, separation of aggregates.

Moreover, Patent literature No. 1 describes a media into which a ligand is introduced by graft polymerization. The present literature describes a media obtained by allowing the graft polymerization of a portion having a negative charge and a hydrophobic portion on a media has excellent adsorption performance even in a higher salt concentration (for example, under a 150 mM sodium chloride (NaCl) solution).

The electrical conductivity of the solution having the NaCl concentration of 150 mM is known to correspond to the electrical conductivity of the cell culture supernatant (for example, Patent literature No. 1). Thus, the media having good adsorption performance at the electrical conductivity (approximately 15 mS/cm) of the culture supernatant is referred to as a media having salt tolerance. The chromatography media having the salt tolerance is particularly beneficial in purifying a biomedicine containing a substance produced by a living organism, such as protein. As described above, when conventional ion exchange chromatography is used, salt removal or dilution of a sample solution is necessary to be performed before injection into a column. However, if a salt-tolerant chromatography media is used, purification can be performed without performing the salt removal.

Patent literature No. 2 discloses a chromatography media having polyamine as a ligand, and use thereof for purification of a blood coagulation factor.

Moreover, Patent literature No. 3 discloses a chromatography media in which polyallylamine is used as a ligand. The media has excellent capability of binding protein such as bovine serum albumin (BSA) even under a solution having a high concentration of sodium chloride (for example, 0.25 M or more).

Moreover, Patent literature No. 4 discloses a membrane having a surface covered with a crosslinked polymer of polyallylamine. The present literature indicates that such a membrane can achieve BSA binding capacity of 60 g/L in a solution having a flow rate of 29 CV/min/bar and a sodium chloride concentration of 200 mM.

Further, Patent literature No. 5 discloses a chromatography media having a ligand containing allylamine or polyallylamine in which the allylamine or the polyallylamine is further modified with any other functional group. However, Patent literature No. 5 is silent to salt tolerance of the chromatography media.

### CITATION LIST

### Patent Literature

Patent literature No. 1: JP 2010-528271 A.

Patent literature No. 2: JP H7-22702 B.

Patent literature No. 3: WO 2009/145722 A.

Patent literature No. 4: JP 5031695 B.

Patent literature No. 5: WO 2012/151352 A.

### Non-Patent Literature

Non-patent literature No. 1: Journal of Chromatography A 1217 (2010), 216-224.

### SUMMARY OF INVENTION

### Technical Problem

Under a background as described above, a chromatography media having salt tolerance and desirable adsorption characteristics is required.

### Solution to Problem

The present inventors have diligently continued to conduct study in order to solve the problems as described above. As a result, the present inventors have found that a chromatography media obtained by adding polyamine to a base media involving porous particles and then modifying part of amino groups in the polyamine with a hydrophobic group has excellent salt tolerance and excellent adsorption characteristics, and thus have completed the invention. More specifically, the invention includes the items described below.

Item 1. A chromatography media containing

a base media involving porous particles and

polyamine bonded with the base media,

wherein 20 to 40% of amino groups in the polyamine is modified with a hydrophobic group.

Item 2. The chromatography media according to item 1, wherein the polyamine is selected from the group of polyallylamine, polyvinylamine, chitosan, polylysine, polyguanidine and polyornithine.

Item 3. The chromatography media according to item 2, wherein the polyamine is polyallylamine.

Item 4. The chromatography media according to item 3, wherein the weight average molecular weight of the polyallylamine is 5,000 to 15,000.

Item 5. The chromatography media according to any one of items 1 to 4, wherein the hydrophobic group has any one of structure represented by general formulas (1) to (3) below:

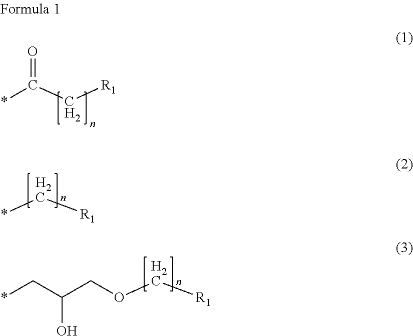

(wherein, in formulas (1) to (3),

n is an integer from 0 to 8,

R₁ is a phenyl group when n is an integer from 0 to 3, and H or a phenyl group when n is an integer from 4 to 8, and

an asterisk (*) represents a site to be bonded with one of the amino groups in the polyamine.)

Item 6. The chromatography media according to item 5, wherein the hydrophobic group has structure represented by the general formula (1).

Item 7. The chromatography media according to item 6, wherein n is an integer from 4 to 8, and R₁ is H in the general formula (1).

Item 8. The chromatography media according to item 6, wherein n is an integer from 0 to 8, and R₁ is a phenyl group in the general formula (1).

Item 9. The chromatography media according to any one of items 1 to 4, wherein the hydrophobic group is derived from a compound selected from the group of valeric anhydride, caproic anhydride, enanthic anhydride, caprylic anhydride, pelargonic anhydride, benzoic anhydride, butyl glycidyl ether and phenyl glycidyl ether.

Item 10. The chromatography media according to item 9, wherein the hydrophobic group is derived from valeric anhydride or benzoic anhydride.

Item 10-1. The chromatography media according to anyone of items 1 to 4, wherein the hydrophobic group has structure represented by general formula (4) or (5) below:

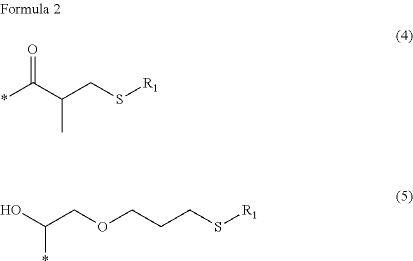

(wherein, in formulas (4) and (5),

R₁ is a heterocyclic group, and

an asterisk (*) represents a site to be bonded with one of the amino groups in polyamine.)

Item 10-2. The chromatography media according to item 10-1, wherein the heterocyclic group contains a nitrogen atom.

Item 10-3. The chromatography media according to item 10-2, wherein a heterocycle in the heterocyclic group is selected from the group of pyridine, imidazole, benzimidazole, pyrazole, imidazoline, pyrazine, indole, isoindole, quinoline, isoquinoline and quinoxaline.

Item 10-4. The chromatography media according to anyone of items 1 to 10-3, wherein the porous particles are crosslinked cellulose particles.

Item 10-5. The chromatography media according to anyone of items 1 to 10-4, wherein a particle size of the porous particles is 10 to 500 micrometers.

Item 10-6. The chromatography media according to anyone of items 1 to 10-5, wherein a Kay value of the porous particles is in the range of 0.15 to 0.6 when standard polyethylene oxide having a weight average molecular weight of 1.5×10⁵ Da is used as a sample, and pure water is used as a mobile phase.

Item 11. The chromatography media according to any one of items 1 to 10-6, wherein static binding capacity of bovine serum albumin per 1 milliliter of the chromatography media is 60 milligrams or more under a 0.2 M NaCl solution.

Item 12. A protein purification method, comprising performing isolation and purification of a protein-containing sample by using the chromatography media according to any one of items 1 to 11.

Item 13. The protein purification method according to item 12, wherein the isolation and purification are performed under a 0.15 to 0.4 M NaCl solution.

### Advantageous Effects of Invention

According to the invention, a chromatography media having excellent salt tolerance, adsorption characteristics and so forth can be provided.

### DESCRIPTION OF EMBODIMENTS

Embodiments of the invention are described in detail below.

A chromatography media of the invention contains

a base media involving porous particles and

polyamine bonded with the base media,

wherein, 20 to 40% of the amino groups in the polyamine is modified with a hydrophobic group.

The chromatography media of the invention contains the polyamine as a ligand and in which part of the amino groups in the polyamine is modified with the hydrophobic group, and therefore has excellent salt tolerance. In particular, the excellent salt tolerance can be achieved by adjusting a ratio of modification with the hydrophobic group for the amino groups in the polyamine to approximately 20 to 40%, while holding ion exchange capacity of the chromatography media. The chromatography media of the invention develops excellent adsorption characteristics not only under conditions of a low salt concentration but also under conditions of a high salt concentration, and therefore can be reasonably stated to be usable in a salt concentration in a wide range.

As described above, a media having good adsorption characteristics in the presence of a salt solution (NaCl solution having approximately 0.15 M) showing electrical conductivity equal to or higher than the electrical conductivity of a general cell culture supernatant is referred to as a media having salt tolerance. The chromatography media of the invention can develop desirable adsorption characteristics obviously under absence of NaCl, and also under a NaCl solution having approximately 0.15 M or more. Further, even under a NaCl solution having approximately 0.2 M or more, for example, under a NaCl solution having approximately 0.2 to approximately 0.4 M, the desirable adsorption characteristics can be obtained. Specifically, when the chromatography media of the invention is used, an amount of bovine serum albumin adsorption in approximately 60 milligrams or more per 1 milliliter of the chromatography media can be obtained under the NaCl solution of approximately 0.2 M.

“Adsorption amount of bovine serum albumin (BSA)” herein is expressed in terms of a value of static binding capacity (namely, saturated adsorption amount) measured in a batch process. Details of a measurement method are as described in Examples described later. Moreover, “1 milliliter of chromatography media” means a chromatography media in an amount constituting 1 milliliter in a volume upon packing the media into a column in a wet gel state.

As described above, the chromatography media of the invention has the salt tolerance, and therefore excellent in capability of being directly applied to chromatography without performing salt removal or dilution of a sample in a culture liquid. In particular, the chromatography media is useful in isolation and purification of protein, and accordingly can be used for purification of a biomedicine or the like. Specifically, the chromatography media can be used in isolation and purification of a vaccine, an antibody, an enzyme, a blood coagulation factor or the like.

Moreover, the chromatography media of the invention has structure of containing as the ligand the polyamine and the hydrophobic group as is different from a conventional media, and therefore is expected to have selectivity never before.

Each constituent of the chromatography media of the invention is described in the order below.

### 1. Base Media

A chromatography media generally has structure in which a ligand is bonded with a base media. The base media in the invention involves porous particles, and the porous particles are modified with a functional group (for example, a hydroxy group and a carbamoyl group) for introducing polyamine as the ligand thereinto. The porous particles to be used are not limited, as long as the particles can be modified with such a functional group. Specific examples preferably include a polysaccharide such as agarose, dextran, starch, cellulose, pullulan, chitin, chitosan, triacetyl cellulose and diacetyl cellulose, and a derivative thereof, and an organic polymer such as polyacrylamide, polymethacrylamide, polyacrylate, polymethacrylate, polyalkylvinyl ether and polyvinyl alcohol. The porous particles preferably form crosslinked structure in view of capability of ensuring mechanical strength. Among the porous particles, crosslinked cellulose particles in which a skeleton of the cellulose particles is reinforced by a crosslinking reaction is further preferably used.

The crosslinked cellulose particles are not particularly limited if the particles can be used as the base media for the chromatography media. The cellulose used as a raw material may be crystalline cellulose or amorphous cellulose, but the crystalline cellulose is preferred due to high strength.

Specific examples of the crosslinked cellulose particles that can be preferably used in the invention include a porous cellulose gel disclosed in JP 2009-242770 A. The porous cellulose gel disclosed in JP 2009-242770 A is obtained by a method comprising a process of performing continuous dropwise addition or split addition of a crosslinking agent in an amount of approximately 4 to 12 times the number of moles of a cellulose monomer and alkali in an amount of approximately 0.1 to 1.5 times the number of moles of the crosslinking agent, over approximately 3 hours or more, to a suspension of non-crosslinked cellulose particles in the presence of at least one kind of an inorganic salt selected from the group of hydrochloride, sulfate, phosphate and borate in an amount of approximately 6 to 20 times the number of moles of the cellulose monomer. The thus obtained crosslinked cellulose particles have high mechanical strength and can be used under chromatography conditions in which a flow rate is high, and thus can give a cation exchange chromatography media having high productivity. Here, “cellulose monomer” means a glucose unit being a constitutional unit of cellulose. Moreover, the number of moles of the cellulose monomer (namely, degree of polymerization) is calculated based on an amount obtained by subtracting moisture from one unit of glucose (namely, dry weight of cellulose) (the molecular weight of 162 is taken as 1 mol).

A shape of the crosslinked cellulose particles is not particularly limited, but is preferably spherical in view of high mechanical strength, excellent gel sedimentation properties and capability of preparation of a uniform packed bed. In the above case, sphericity of the crosslinked cellulose particles is preferably approximately 0.8 to approximately 1.0. Here, “sphericity” means a ratio of minor axis to major axis (minor axis/major axis) of the cellulose particles.

Spherical cellulose particles can be easily obtained, for example, by dissolving crystalline cellulose or cellulose having a crystalline region and an amorphous region into a solvent to regenerate the cellulose. Specific examples of a method for manufacturing the spherical cellulose particles include a method through acetate ester as described in JP S55-39565 B and JP S55-40618 B, a method for manufacturing the cellulose from a solution containing calcium thiocyanate as described in JP S63-62252 B, a method for manufacturing the cellulose from a solution containing paraformaldehyde and dimethylsulfoxide as described in JP S59-38203 A, and a method for manufacturing the cellulose from a cellulose solution in which the cellulose is dissolved into lithium chloride-containing amide as described in JP 3663666 B. Moreover, spherical crosslinked cellulose particles can be obtained by crosslinking the spherical cellulose particles.

A particle size of the porous particles used in the invention is preferably approximately 10 to 500 micrometers, further preferably approximately 30 to 200 micrometers, and particularly preferably approximately 50 to 150 micrometers. Moreover, a mean particle size is preferably approximately 30 to 1,000 micrometers, further preferably approximately 40 to 200 micrometers, and particularly preferably approximately 50 to 100 micrometers. Here, “particle size” means an actually measured value of the particle size of individual porous particles, and “mean particle size” means a mean value calculated based on the particle size.

The particle size and the mean particle size of the porous particles herein can be measured using a laser diffraction scattering particle size distribution analyzer. In the analyzer, a group of particles is irradiated with a laser beam to determine a particle size distribution from a diffracted and scattered light intensity distribution pattern emitted from the group of particles, and then the particle size and mean particle size are calculated based thereon. As a specific analyzer, Laser Diffraction Scattering Particle Size Distribution Analyzer LA-950 (made by HORIBA, Ltd.) or the like can be used.

Alternatively, the particle size can also be measured using an image photographed with an optical microscope. Specifically, the particle size on the image is measured using a caliper or the like, and an original particle size is determined using photographing magnification. Then, from a value of each particle size determined from an optical microscope photograph, the mean particle size is calculated using the following formula:

Volume mean particle size (MV)=Σ(nd⁴)/Σ(nd³)

(In the formula, d represents a value of each particle size determined from the optical microscope photograph, and n represents the number of measured particles.)

Porosity of the porous particles can be featured by pore size characteristics. One of indices indicating the pore size characteristics includes a gel partition coefficient Kay. The pore size influences physical strength of the particles and diffusivity of an objective material being a purification target in the porous particles. Accordingly, a difference is caused, depending on the pore size, in a flow rate of a liquid passing through an inside of the porous particles and dynamic adsorption capacity of the porous particles. Therefore, a design of the porous particles to be the pore size according to an intended purpose is required. In particular, from a viewpoint of the dynamic adsorption capacity, the gel partition coefficient Kav of the porous particles is preferably in the range of approximately 0.15 to approximately 0.6, further preferably in the range of approximately 0.2 to approximately 0.55 and particularly preferably in the range of approximately 0.3 to approximately 0.5, when standard polyethylene oxide having a weight average molecular weight of 1.5×10⁵ Da is used as a sample and pure water is used as a mobile phase.

In the invention, the porous particles having the pore size in which the gel partition coefficient in the range described above is obtained are preferably used from a viewpoint of adsorption characteristics. When the crosslinked cellulose particles are used as the porous particles, the gel partition coefficient Kav thereof can be adjusted by controlling a concentration of dissolved cellulose during formation of the particles, for example.

The gel partition coefficient Kav can be determined from a relationship between retention volume and column volume when a standard material (polyethylene oxide, for example) having a specific molecular weight was used as a sample, according to the following formula:

Kav=(Ve−V₀)/(Vt−V₀).

(In the formula, Ve represents retention volume (mL) of a sample, Vt represents empty column volume (mL), and V₀ represents blue dextran retention volume (mL).)

A specific method for measuring the gel partition coefficient Kav is described in Seibutsukagaku Jikkenho (Biochemistry Experimental Method) 2 “Gel chromatography,” first edition, authored by L. Fischer (Tokyo Kagaku Dojin), for example.

### 2. Ligand

The chromatography media of the invention contains polyamine as a ligand, and approximately 20 to approximately 40% of the amino groups in the polyamine are modified with the hydrophobic group.

### (2-1) Polyamine

First, polyamine is described.

The polyamine is a generic term for aliphatic hydrocarbon in which a plurality of primary amino groups are bonded. The polyamine used in the invention is not particularly limited, if the polyamine can be bonded with a functional group on a base media. Specific examples include polyallylamine, polyvinylamine, chitosan, polylysine, polyguanidine and polyornithine. Above all, polyallylamine and polylysine are preferred, and polyallylamine is further preferred.

The weight average molecular weight of the polyamine may be approximately 300,000 or less, preferably approximately 1,000 to approximately 100,000, further preferably approximately 3,000 to approximately 50,000, and particularly preferably approximately 5,000 to approximately 15,000. When the polyallylamine is used, the weight average molecular weight maybe approximately 150,000 or less, preferably approximately 1,000 to approximately 100,000, further preferably approximately 3,000 to approximately 50,000, and particularly preferably approximately 5,000 to approximately 15,000.

A method for adding the polyamine to the base media is not particularly limited, and addition thereof can be performed by a publicly known method. For example, the addition can be performed, under predetermined conditions, by stirring a solution containing the polyamine and the porous particles modified with a functional group (for example, a hydroxy group and a carbamoyl group) with which the polyamine can be bonded.

Alternatively, the polyamine may be formed thereon by allowing graft polymerization of monomers on the base media. In the above case, a compound containing a primary amino group may be used as the monomer, or the polyamine may be obtained by allowing graft polymerization of a monomer having a group reactive with amine, such as glycidyl methacrylate, on the base media, and then allowing the resulting material to react with ammonia.

### (2-2) Hydrophobic Group

A hydrophobic group is not particularly limited, as long as the hydrophobic group is bonded with the amino group in the polyamine and has hydrophobicity, but the hydrophobic group ordinarily used in a hydrophobic chromatography media is preferred. Specific examples of such a hydrophobic group include a group containing a saturated alkyl group and/or a phenyl group. As the saturated alkyl group, a linear saturated alkyl group is preferred, a linear C4 to C8 saturated alkyl group is further preferred, and a n-butyl group is particularly preferred.

Specific examples of structure of a preferred hydrophobic group include structure represented by any one of the following general formulas (1) to (3).

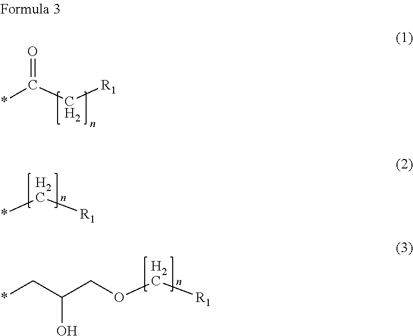

(In formulas (1) to (3),

n is an integer from 0 to 8,

R₁ is a phenyl group when n is an integer from 0 to 3, H or a phenyl group when n is an integer from 4 to 8, and

an asterisk (*) represents a site to be bonded with one of the amino groups in the polyamine.)

In other words, n is the integer from 0 to 8 when R₁ is the phenyl group, and is the integer from 4 to 8 when R₁ is H.

A carbon atom in the structure represented by formulas (1) to (3) described above may have a substituent such as an alkyl group and an alkoxy group.

In the structure represented by the general formulas (1) to (3), the structure represented by the general formula (1) is further preferred.

A mode of bonding of the hydrophobic group with the amino group is not particularly limited, if the mode is a covalent bond. Specifically, the mode may be an amide bond formed by a reaction between acid anhydride, acid chloride or active ester and the amino group, for example, or a carbon-nitrogen bond formed by a reaction between an epoxy compound or halogenide, and the amino group.

Specific examples of the compound for introducing the hydrophobic group described above thereinto include valeric anhydride, caproic anhydride, enanthic anhydride, caprylic anhydride, pelargonic anhydride, benzoic anhydride, butyl glycidyl ether and phenyl glycidyl ether. More specifically, the hydrophobic group in the invention is conveniently a group derived from the above compounds. Here, the hydrophobic group can be bonded with the amino group of the polyamine by allowing the above compounds to react with the polyamine. In the compounds described above, valeric anhydride and benzoic anhydride are further preferred. The acid anhydride is preferred in view of efficient progress of the reaction with the polyamine under mild conditions.

A method upon modifying the amino group in the polyamine with the hydrophobic group is not particularly limited, but modification thereof can be performed by a publicly known method. For example, the modification can be performed by stirring a solution containing the polyamine and the compound for introducing the hydrophobic group under predetermined conditions.

Structure of the hydrophobic group may be represented by the following general formula (4) or (5).

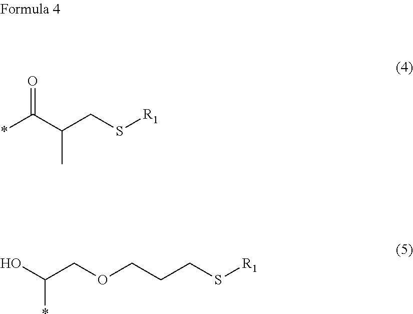

(In formulas (4) and (5),

R₁ is a heterocyclic group, and

an asterisk (*) represents a site to be bonded with one of the amino groups in the polyamine.)

The heterocyclic group R₁ is not particularly limited, but is preferably a heterocyclic group containing a nitrogen atom, and further preferably an aromatic heterocyclic group having a nitrogen atom. Specific examples of the heterocycle in the heterocyclic group include pyridine, imidazole, benzimidazole, pyrazole, imidazoline, pyrazine, indole, isoindole, quinoline, isoquinoline and quinoxaline, and pyridine, imidazole and benzimidazole are preferred.

A carbon atom in the heterocyclic group may have a substituent. Specific examples of the substituent include an alkyl group and an alkoxy group, and a methyl group and a methoxy group are preferred.

In order to introduce the hydrophobic group represented by formula (4) described above into the polyamine, for example, a methacryl group is bonded with the amino group in the polyamine, and further a heterocycle-containing group is bonded with the methacryl group. The methacryl group can be introduced thereinto by allowing the amino group in the polyamine to react with acid chloride of methacrylic anhydride, methacrylic acid or an active ester compound derived from methacrylic acid, or the like. Moreover, the heterocycle-containing group can be introduced thereinto by allowing the heterocyclic group-containing compound to react with the methacryl group bonded with the amino group. The heterocyclic group-containing compound contains the heterocyclic group and the thiol group, for example, and in the above case, the thiol group reacts with the methacryl group.

In order to introduce the hydrophobic group represented by formula (5) described above into the polyamine, an allyl group is bonded with the amino group in the polyamine, and further a heterocycle-containing group is bonded with the allyl group, for example. The allyl group can be introduced thereinto by allowing the amino group in the polyamine to react with a compound having both a functional group to be bonded with the amino group and the allyl group (for example, allylglycidylether). Moreover, the heterocycle-containing group can be introduced thereinto by allowing the heterocyclic group-containing compound to react with the allyl group bonded with the amino group. The heterocyclic group-containing compound contains the heterocyclic group and the thiol group, for example, and in the above case, the thiol group reacts with the allyl group or the functional group derived from the allyl group.

The heterocyclic group-containing compound used in introducing the hydrophobic group represented by formula (4) and (5) described above is not particularly limited, as long as the compound can introduce the heterocyclic group, but the compound containing the heterocyclic group and the thiol group is preferred. Specific examples of such a compound include 2-mercaptoethylpyridine, 2-mercaptobenzimidazole, 2-mercapto-4-methylimidazole and 2-mercapto-4,5-methylimidazole.

In the invention, approximately 20 to approximately 40% of the amino groups in the polyamine are modified with the hydrophobic group, more specifically, the ratio of modification of the amino group is approximately 20 to approximately 40%. The ratio of modification of the amino group is expressed in terms of a value based on the total number of primary amino groups existing in the polyamine. For example, when 100 pieces of primary amino groups exist in the polyamine, the values of approximately 20 to approximately 40% mean that approximately 20 to approximately 40 pieces of amino groups thereof are modified with the hydrophobic group. The ratio of modification of the amino group is preferably approximately 20 to approximately 40%, and further preferably approximately 20 to approximately 30%. Upon allowing the polyamine to react with the compound for introducing the hydrophobic group, the ratio of modification of the amino group can be adjusted to be within the range described above by adjusting an amount of the compound for introducing the hydrophobic group. The ratio of modification of the amino group can be calculated by measuring ion exchange capacity of the chromatography media before and after introducing the hydrophobic group, and comparing the values.

According to another embodiment of the invention, a chromatography media is provided in which static binding capacity to the bovine serum albumin per 1 milliliter of chromatography media is approximately 60 milligrams or more under a 0.2 M NaCl solution. The chromatography media satisfying such requirements can be evaluated to have sufficient salt tolerance, and can be advantageously used in purification of the biomedicine.

According to another embodiment of the invention, a method of purifying protein comprising performing isolation and purification of a protein-containing sample by using the chromatography media described above is provided. The isolation and purification can be performed satisfactorily under a NaCl solution having approximately 0.15 M or higher.

It will be apparent to those skilled in the art that various modifications and variations can be made in the invention and specific examples provided herein without departing from the spirit or scope of the invention. Thus, it is intended that the invention covers the modifications and variations of this invention that come within the scope of any claims and their equivalents.

The following examples are for illustrative purposes only and are not intended, nor should they be interpreted to, limit the scope of the invention.

### EXAMPLES

The invention is described in detail by way of Examples below, but the content of the invention is not limited by the Examples.

### Example 1

### Manufacture of 6% Spherical Cellulose Particles (Hydrous)

Then, 6% spherical cellulose particles were manufactured according to the following procedures. Here, cellulose particles manufactured when a concentration of crystalline cellulose is 6% by weight in process (1) below are referred to as “6% spherical cellulose particles.”

(1) To 100 g of 60 wt % calcium thiocyanate aqueous solution, 6.4 g of crystalline cellulose (trade name: Ceolus PH101, made by Asahi Kasei Chemicals Corporation) was added, and the resulting mixture was heated at 110 to 120° C. and dissolved with each other.

(2) As a surfactant, 6 g of sorbitan monooleate was added to the resulting solution. The resulting mixture was added dropwise to 480 mL of o-dichlorobenzene preheated at 130 to 140° C., and the resulting mixture was stirred at 200 to 300 rpm to obtain a dispersion.

(3) Next, the resulting dispersion was cooled to 40° C. or lower, and poured into 190 mL of methanol to obtain a suspension of particles.

(4) The resulting suspension was filtered and fractionated, and particles were washed with 190 mL of methanol. The above washing operation was performed several times.

(5) The particles were further washed with a plenty of water to obtain spherical cellulose particles.

(6) Next, the spherical cellulose particles were sieved through a sieve having 53 μm to 125 μm according to a JIS standard sieve specification to obtain 6% spherical cellulose particles having a desired particle size (particle size: 50 to 150 μm, mean particle size: approximately 100 μm) (hydrous: cellulose dissolved concentration: 6%).

In addition, the mean particle size here was measured using an image photographed by an optical microscope. Specifically, the particle size on the image was measured using a caliper, and an original particle size was determined from photographing magnification. Then, the mean particle size was calculated from values of individual particle size values determined from the optical microscope image, according to the following formula.

Volume mean particle size (MV)=Σ(nd⁴)/Σ(nd³)

(In the formula, d represents a value of a particle size of each particle determined from the optical microscope image, and n represents the number of particles measured.)

### Manufacture of Crosslinked 6% Cellulose Particles

Crosslinked 6% cellulose particles were manufactured by allowing a crosslinking reaction of the 6% spherical cellulose particles manufactured as described above. The procedures are as described below.

(1) To 100 g of 6% spherical cellulose particles (hydrous) obtained as described above, 121 g of pure water was added, and the resulting mixture was warmed while the mixture was stirred. When temperature reached 30° C., 3.3 g of 45 wt % NaOH aqueous solution and 5.5 g of NaBH₄ were added thereto, and the resulting mixture was further warmed and stirred. An initial alkali concentration here was 0.69% (w/w).

(2) After 30 minutes, 60 g of Na₂SO₄ was added to the resulting reaction mixture warmed at 40° C., and dissolved thereinto. At the time point at which temperature reached 50° C., stirring was further continued for two hours while the temperature was maintained at 50° C.

(3) While stirring of the resulting mixture was continued at 50° C., an amount obtained by equally dividing into 25 each of 48 g of 45 wt % NaOH aqueous solution and 50 g of epichlorohydrin was added thereto every 15 minutes over approximately 6 hours.

(4) After addition completion, the resulting mixture was allowed to react with each other at a temperature of 50° C. for 16 hours.

(5) After the resulting reaction mixture was cooled to 40° C., 2.6 g of acetic acid was added thereto, and thus the resulting mixture was neutralized.

(6) The resulting reaction mixture was filtered to collect cellulose particles, and the cellulose particles were subjected to filtration and washing with pure water to obtain crosslinked 6% cellulose particles.

A mean particle size and a Kav value of the crosslinked 6% cellulose particles obtained were measured as described below.

### (Measurement of Mean Particle Size)

When the mean particle size was analyzed using Laser Diffraction Scattering Particle Size Distribution Analyzer LA-950 (made by HORIBA, Ltd.), the mean particle size was 85 μm.

### (Measurement of Kav Value)

The gel partition coefficient Kav was calculated from a relationship between retention volume and column volume thereof by using a standard polyethylene oxide having a weight average molecular weight of 1.5×10⁵ Da as a sample, according to the following formula. In addition, pure water was used as a mobile phase.

Kav=(Ve−V₀)/(Vt−V₀)

(In the formula, Ve represents retention volume (mL) of the sample, Vt represents empty column volume (mL), and V₀ represents retention volume (mL) of blue dextran.)

The gel partition coefficient Kav of the crosslinked 6% cellulose particles obtained as described above was 0.38.

### Epoxidation of Crosslinked 6% Cellulose Particles

Then, 500 g of the crosslinked 6% cellulose particles obtained as described above was put in a 2 L separable flask, and 745 g of pure water was added thereto into slurry. Liquid temperature was increased up to 26° C. by a warm bath and 48.7 wt % sodium hydroxide aqueous solution was added over time without exceeding 30° C. in the liquid temperature. Then, 343 g of epichlorohydrin was added thereto and the resulting mixture was stirred at a liquid temperature of 30° C. for 2 hours. After reaction completion, cellulose particles were collected by filtration, collected cellulose particles were washed 10 times with 1 L of pure water to obtain epoxidized cellulose particles.

### Addition of Polyallylamine

In a 1 L separable flask, 150 g of the epoxidized cellulose particles obtained as described above was put, 385 g of PAA-05 (made by Nittobo Medical Co., Ltd.) being a 20% (w/w) aqueous solution of polyallylamine having a weight average molecular weight of 5,000 was added thereto, and the resulting mixture was stirred at 45° C. for 18 hours. After reaction completion, cellulose particles were collected by filtration, collected cellulose particles were washed 10 times with 300 mL of pure water to obtain polyallylamine-added cellulose particles. Ion exchange capacity of the polyallylamine-added cellulose particles was 0.22 mmol/mL. A method for measuring the ion exchange capacity is as described later.

Modification with Hydrophobic Group

Then, 15 g of the polyallylamine-added cellulose particles obtained as described above was washed 5 times with 45 mL of methanol. The methanol-washed particles were put in a 50 mL centrifugal tube, and 15 mL of methanol was added thereto into slurry. Then, 0.30 g of valeric anhydride and 0.30 g of tris(hydroxymethyl)aminomethane were added thereto, and the resulting mixture was stirred at 25° C. for 24 hours. After reaction completion, cellulose particles were collected by filtration and the collected cellulose particles were washed once with 100 mL of methanol and 5 times with 100 mL of pure water to obtain polyallylamine-added cellulose particles modified with a hydrophobic group.

### Comparative Example 1

Polyallylamine-added cellulose particles were manufactured in a manner similar to the method in Example 1.

Polyallylamine-added cellulose particles modified with a hydrophobic group were obtained in a manner similar to the method in Example 1 except that an amount of valeric anhydride to be added was changed to 0.60 g and an amount of tris(hydroxymethyl)aminomethane to be added was changed to 0.60 g.

### Comparative Example 2

Polyallylamine-added cellulose particles were manufactured in a manner similar to the method in Example 1.

Polyallylamine-added cellulose particles modified with a hydrophobic group were obtained in a manner similar to the method in Example 1 except that an amount of valeric anhydride to be added was changed to 1.40 g and an amount of tris(hydroxymethyl)aminomethane to be added was changed to 1.41 g.

### Example 2

Polyallylamine-added cellulose particles were manufactured in a manner similar to the method in Example 1.

Then, 15 g of the polyallylamine-added cellulose particles obtained was washed 5 times with 45 mL of methanol. The methanol-washed particles were put in a 50 mL centrifugal tube, and 15 mL of methanol was added thereto into slurry. Then, 0.35 g of benzoic anhydride and 0.35 g of tris(hydroxymethyl)aminomethane were added thereto, and the resulting mixture was rotatively stirred at 25° C. for 24 hours. After reaction completion, cellulose particles were collected by filtration and collected cellulose particles were washed once with 100 mL of methanol and 5 times with 100 mL of pure water to obtain polyallylamine-added cellulose particles modified with the hydrophobic group.

### Comparative Example 3

Polyallylamine-added cellulose particles were manufactured in a manner similar to the method in Example 1.

Polyallylamine-added cellulose particles modified with a hydrophobic group were obtained in a manner similar to the method in Example 2 except that an amount of benzoic anhydride to be added was changed to 0.72 g and an amount of tris(hydroxymethyl)aminomethane to be added was changed to 0.73 g.

### Comparative Example 4

Polyallylamine-added cellulose particles were manufactured in a manner similar to the method in Example 1.

Polyallylamine-added cellulose particles modified with a hydrophobic group were obtained in a manner similar to the method in Example 2 except that an amount of benzoic anhydride to be added was changed to 1.66 g and an amount of tris(hydroxymethyl)aminomethane to be added was changed to 1.67 g.

### Comparative Example 5

Polyallylamine-added cellulose particles were manufactured in a manner similar to the method in Example 1.

Then, 15 g of the polyallylamine-added cellulose particles obtained was washed 5 times with 45 mL of methanol. The methanol-washed particles were put in a 50 mL centrifugal tube, and 15 mL of methanol was added thereto into slurry. Then, 0.20 g of acetic anhydride and 0.15 g of tris(hydroxymethyl)aminomethane were added thereto, and the resulting mixture was rotatively stirred at 25° C. for 24 hours. After reaction completion, cellulose particles were collected by filtration and collected cellulose particles were washed once with 100 mL of methanol and 5 times with 100 mL of pure water to obtain polyallylamine-added cellulose particles modified with the hydrophobic group.

### Comparative Example 6

Polyallylamine-added cellulose particles were manufactured in a manner similar to the method in Example 1.

Polyallylamine-added cellulose particles modified with a hydrophobic group were obtained in a manner similar to the method in Comparative Example 5 except that an amount of acetic anhydride to be added was changed to 0.35 g and an amount of tris(hydroxymethyl)aminomethane to be added was changed to 0.35 g.

### Comparative Example 7

Polyallylamine-added cellulose particles were manufactured in a manner similar to the method in Example 1.

Polyallylamine-added cellulose particles modified with a hydrophobic group were obtained in a manner similar to the method in Comparative Example 5 except that an amount of acetic anhydride to be added was changed to 0.80 g and an amount of tris(hydroxymethyl)aminomethane to be added was changed to 0.81 g.

### Example 3

Epoxidized cellulose particles were manufactured in a manner similar to the method in Example 1.

Then, 150 g of the epoxidized cellulose particles obtained were put in a 1 L separable flask, 280 g of PAA-15C (made by Nittobo Medical Co., Ltd.) being a 15% (w/w) aqueous solution of polyallylamine having a weight average molecular weight of 15,000 was added thereto, and the resulting mixture was stirred at 45° C. for 18 hours. After reaction completion, cellulose particles were collected by filtration and collected wet particles were washed 10 times with 300 mL of pure water to obtain polyallylamine-added cellulose particles. Ion exchange capacity of the polyallylamine-added cellulose particles was 0.29 mmol/mL. A method of measuring the ion exchange capacity is as described later.

Modification with Hydrophobic Group

Then, 15 g of the polyallylamine-added cellulose particles obtained as described above was washed 5 times with 45 mL of methanol. The methanol-washed particles were put in a 50 mL centrifugal tube, and 15 mL of methanol was added thereto into slurry. Then, 0.37 g of valeric anhydride and 0.37 g of tris(hydroxymethyl)aminomethane were added thereto, and the resulting mixture was rotatively stirred at 25° C. for 24 hours. After reaction completion, cellulose particles were collected by filtration and collected cellulose particles were washed once with 100 mL of methanol and 5 times with 100 mL of pure water to obtain polyallylamine-added cellulose particles modified with the hydrophobic group.

### Comparative Example 8

Polyallylamine-added cellulose particles were manufactured in a manner similar to the method in Example 3.

Polyallylamine-added cellulose particles modified with a hydrophobic group were obtained in a manner similar to the method in Example 3 except that an amount of valeric anhydride to be added was changed to 0.87 g and an amount of tris(hydroxymethyl)aminomethane to be added was changed to 0.88 g.

### Comparative Example 9

Polyallylamine-added cellulose particles were manufactured in a manner similar to the method in Example 3.

Polyallylamine-added cellulose particles modified with a hydrophobic group were obtained in a manner similar to the method in Example 3 except that an amount of valeric anhydride to be added was changed to 2.50 g and an amount of tris(hydroxymethyl)aminomethane to be added was changed to 2.52 g.

### Example 4

Polyallylamine-added cellulose particles were manufactured in a manner similar to the method in Example 3.

Then, 15 g of the polyallylamine-added cellulose particles obtained as described above was washed 5 times with 45 mL of methanol. The methanol-washed particles were put in a 50 mL centrifugal tube, and 15 mL of methanol was added thereto into slurry. Then, 0.50 g of benzoic anhydride and 0.50 g of tris(hydroxymethyl)aminomethane were added thereto, and the resulting mixture was rotatively stirred at 25° C. for 24 hours. After reaction completion, cellulose particles were collected by filtration and collected cellulose particles were washed once with 100 mL of methanol and 5 times with 100 mL of pure water to obtain polyallylamine-added cellulose particles modified with the hydrophobic group.

### Comparative Example 10

Polyallylamine-added cellulose particles were manufactured in a manner similar to the method in Example 3.

Polyallylamine-added cellulose particles modified with a hydrophobic group were obtained in a manner similar to the method in Example 4 except that an amount of benzoic anhydride to be added was changed to 1.00 g and an amount of tris(hydroxymethyl)aminomethane to be added was changed to 1.01 g.

### Comparative Example 11

Polyallylamine-added cellulose particles were manufactured in a manner similar to the method in Example 3.

Polyallylamine-added cellulose particles modified with a hydrophobic group were obtained in a manner similar to the method in Example 4 except that an amount of benzoic anhydride to be added was changed to 3.00 g and an amount of tris(hydroxymethyl)aminomethane to be added was changed to 3.02 g.

With regard to the chromatography media prepared in Examples and Comparative Examples described above, ion exchange capacity, a ratio of modification of the amino group, and an adsorption amount of BSA were measured. The measurement method is as described below and the measurement results are shown in Table 1.

### Measurement Method

### (1) Ion Exchange Capacity

Then, 1 mL of chromatography media (chromatography media in an amount constituting 1 mL in volume upon packing the media into a column in a wet gel state) was put in a 50 mL Erlenmeyer flask. Thereto, 50 mL of 0.01 mol/L hydrochloric acid aqueous solution was added, and the flask was gently shaken. After allowing the flask to stand at room temperature for 1 hour, 10 mL of supernatant was weighed in a 50 mL beaker by a volumetric pipette. Thereto, a phenolphthalein solution was added, and the resulting solution was titrated with a 0.01 mol/L sodium hydroxide aqueous solution. An adsorption amount of hydrochloric acid was calculated from a titer to determine ion exchange capacity per 1 mL of chromatography media.

### (2) Ratio of Modification of Amino Group

With regard to the chromatography media before and after the amino group in the polyamine bonded with the base media was modified with the hydrophobic group (hereinafter, also referred to as “before modification with hydrophobic group” and “after modification with hydrophobic group”), ion exchange capacity was measured by the method described above, respectively. The ratio of modification of the amino group in the polyamine was calculated using values of measured ion exchange capacity, from the following equation.

$$Ratio of modification ( \% ) = \frac{( \begin{matrix}Ion exchange capacity before \\ modification with hydrophobic group\end{matrix} ) - ( \begin{matrix}Ion exvhange capacity after \\ modification with hydrophobic group\end{matrix} )}{Ion exchange capacity after modification with hydrophobic group} \times 100 \qquad Equation 1$$

### (3) Adsorption Amount of BSA (Static Binding Capacity)

Then, 0.1 g of chromatography media was put in a 15 mL centrifugal tube, 10 mL of 3 mg/mL BSA solution dissolved into a 50 mM Tris-HCl buffer (pH 8.5) was added thereto, and the resulting mixture was rotatively stirred at room temperature for 24 hours. Then, a solid and a liquid were separated by centrifugal separation and absorbance (280 nm) of a supernatant was measured. An adsorption amount of BSA was determined from a difference between absorbance (280 nm) of the BSA solution previously measured and the absorbance of the supernatant measured as described above. Finally, in consideration of weight and volume of the chromatography media, the adsorption amount of BSA per 1 mL of chromatography media was calculated.

In the above description, three kinds of BSA solutions shown below were arranged for each of Examples and Comparative Examples, and a test was conducted for each.

NaCl concentration in BSA solution=0 M (electrical conductivity: 1.5 mS/cm).

NaCl concentration in BSA solution=0.1 M (electrical conductivity: 11.7 mS/cm).

NaCl concentration in BSA solution=0.2 M (electrical conductivity: 21.1 mS/cm).

TABLE 1

| | Weight | Compound | | | Adsorption amount | | |
|---|---|---|---|---|---|---|---|
| | average | for | Ion | | of BSA | | |
| | molecular | introducing | exchange | Ratio of | (mg/mL) | | |
| | weight of | hydrophobic | capacity | modification | 0M | 0.1M | 0.2M |
| | polyallylamine | group | (mmol/mL) | (%) | NaCl | NaCl | NaCl |
| Example 1 | 5,000 | Valeric | 153 | 30 | 96 | 73 | 60 |
| | | anhydride | | | | | |
| Comparative | 5,000 | Valeric | 78 | 64 | 61 | 48 | 41 |
| Example 1 | | anhydride | | | | | |
| Comparative | 5,000 | Valeric | 14 | 93 | 18 | 24 | 24 |
| Example 2 | | anhydride | | | | | |
| Example 2 | 5,000 | Benzoic | 146 | 34 | 91 | 76 | 64 |
| | | anhydride | | | | | |
| Comparative | 5,000 | Benzoic | 78 | 65 | 48 | 43 | 37 |
| Example 3 | | anhydride | | | | | |
| Comparative | 5,000 | Benzoic | 19 | 92 | 19 | 24 | 25 |
| Example 4 | | anhydride | | | | | |
| Comparative | 5,000 | Acetic | 132 | 40 | 90 | 61 | 45 |
| Example 5 | | anhydride | | | | | |
| Comparative | 5,000 | Acetic | 59 | 73 | 48 | 24 | 15 |
| Example 6 | | anhydride | | | | | |
| Comparative | 5,000 | Acetic | 7 | 97 | 0 | 3 | 2 |
| Example 7 | | anhydride | | | | | |
| Example 3 | 15,000 | Valeric | 231 | 20 | 123 | 109 | 95 |
| | | anhydride | | | | | |
| Comparative | 15,000 | Valeric | 107 | 63 | 76 | 64 | 53 |
| Example 8 | | anhydride | | | | | |
| Comparative | 15,000 | Valeric | 6 | 98 | 19 | 22 | 24 |
| Example 9 | | anhydride | | | | | |
| Example 4 | 15,000 | Benzoic | 216 | 25 | 109 | 104 | 88 |
| | | anhydride | | | | | |
| Comparative | 15,000 | Benzoic | 123 | 58 | 68 | 62 | 54 |
| Example 10 | | anhydride | | | | | |
| Comparative | 15,000 | Benzoic | 13 | 96 | 17 | 22 | 22 |
| Example 11 | | anhydride | | | | | |

As shown in Table 1, the chromatography media in Examples 1 to 4 have the adsorption amount of BSA in the range of 60 mg or more per 1 mL of chromatography media under the 0.2 M NaCl solution, while the chromatography media held the ion exchange capacity. Accordingly, the chromatography media in Examples 1 to 4 reasonably have the salt tolerance. Such a chromatography media is particularly useful in purification of protein.

### Comparative Example 12

### Addition of Allyl Glycidyl Ether

Polyallylamine-added cellulose particles were manufactured in a manner similar to the method in Example 3.

Then, 30 g of the polyallylamine-added cellulose particles obtained was washed 5 times with 150 mL of methanol. The methanol-washed particles were put in a 150 mL flask, and 60 mL of methanol was added thereto into slurry. Then, a solution prepared by dissolving 1.50 g of allyl glycidyl ether into 1.0 mL of methanol was added thereto, and the resulting mixture was stirred at 25° C. for 24 hours, then, heated to 40° C. and further stirred at 40° C. for 24 hours. After reaction completion, cellulose particles were collected by filtration and collected cellulose particles were washed 3 times with 50 mL of methanol and 5 times with 100 mL of pure water to obtain allyl glycidyl ether-added cellulose particles.

Addition of 4-pyridineethanethiol

Then, 8 g of the allyl glycidyl ether-added cellulose particles obtained was put in a 150 mL flask, and 50 mL of pure water was added thereto into slurry. Then, an inside of the flask was subjected to replacement by a nitrogen gas by repeating vacuuming and replacement by the nitrogen gas 3 times. Next, a solution prepared by dissolving 0.8 g of 4-pyridineethanethiol hydrochloride into 5.0 mL of pure water was put in the flask under a nitrogen gas flow. Next, a solution prepared by dissolving 0.31 g of V-50 (radical initiator) into 5.0 mL of pure water was added to the flask under a nitrogen gas flow. Then, the resulting mixture was heated to 60° C. and stirred 18 hours under a nitrogen atmosphere. After reaction completion, cellulose particles were collected by filtration and collected cellulose particles were washed 4 times with 30 mL of pure water, 3 times with 30 mL of 1 M brine and 5 times with 50 mL of pure water to obtain 4-pyridineethanethiol-added cellulose particles. An N content of the particles obtained was 4.0%, and an S content was 3.2%.

### (Measurement Method)

### (4) Method of Measuring N Content

With regard to measuring an N content in Comparative Example 12, measurement was carried out using Micro Corder JM10 (J-Science Lab Co., Ltd.).

### (5) Method of Measuring S Content

With regard to measuring an S content in Comparative Example 12, measurement was carried out using ICP Emission Spectrometer iCAP6000 (made by Thermo Fisher Scientific Inc.). As pretreatment upon the measurement, 0.02 g of a sample for measurement was decomposed by 15 mL of nitric acid and 2 mL of perchloric acid, and then hydrochloric acid was added to be adjusted in a constant volume of 50 mL.

TABLE 2

| | Weight | | | | Adsorption amount | | |
|---|---|---|---|---|---|---|---|
| | average | | | | of BSA | | |
| | molecular | Compound for | N | S | (mg/mL) | | |
| | weight of | introducing | content | content | 0M | 0.1M | 0.2M |
| | polyallylamine | hydrophobic group | (%) | (%) | NaCl | NaCl | NaCl |
| Comparative | 15,000 | 4-pyridineethanethiol | 4.0 | 3.2 | 118 | 58 | 42 |
| Example 12 | | | | | | | |

Although the invention has been described and illustrated with a certain degree of particularity, it is understood that the disclosure has been made only by way of example, and that numerous changes in the conditions and order of steps can be resorted to by those skilled in the art without departing from the spirit and scope of the invention.

What is claimed is:

1. A chromatography media containing
a base media comprising porous particles and
polyamine bonded with the base media,
wherein 20 to 40% of amino groups in the polyamine is modified with a hydrophobic group.

2. The chromatography media according to claim 1, wherein the polyamine is selected from the group consisting of polyallylamine, polyvinylamine, chitosan, polylysine, polyguanidine and polyornithine.

3. The chromatography media according to claim 2, wherein the polyamine is polyallylamine.

4. The chromatography media according to claim 3, wherein a weight average molecular weight of the polyallylamine is 5,000 to 15,000.

5. The chromatography media according to claim 1, wherein the hydrophobic group has any one of structures represented by general formulas (1) to (3) below: 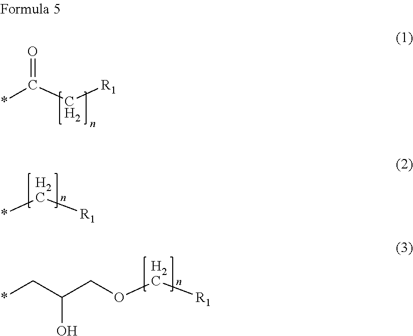
wherein,
n is an integer from 0 to 8,
R₁ is a phenyl group when n is an integer from 0 to 3, and H or a phenyl group when n is an integer from 4 to 8, and
an asterisk (*) represents a site to be bonded with one of the amino groups in the polyamine.

6. The chromatography media according to claim 5, wherein the hydrophobic group has the one of the structures represented by the general formula (1).

7. The chromatography media according to claim 6, wherein n is an integer from 4 to 8 and R₁ is H in the general formula (1).

8. The chromatography media according to claim 6, wherein n is an integer from 0 to 8 and R₁ is a phenyl group in the general formula (1).

9. The chromatography media according to claim 1, wherein the hydrophobic group is derived from a compound selected from the group consisting of valeric anhydride, caproic anhydride, enanthic anhydride, caprylic anhydride, pelargonic anhydride, benzoic anhydride, butyl glycidyl ether and phenyl glycidyl ether.

10. The chromatography media according to claim 9, wherein the hydrophobic group is derived from valeric anhydride or benzoic anhydride.

11. The chromatography media according to claim 1, wherein a static binding capacity of bovine serum albumin per 1 milliliter of the chromatography media is 60 milligrams or more under a 0.2 M NaCl solution.

12. A protein purification method, comprising performing isolation and purification of a protein-containing sample by using the chromatography media according to claim 1.

13. The protein purification method according to claim 12, wherein the isolation and purification are performed under a 0.15 to 0.4 M NaCl solution.
